(19) United States
(12) Patent Application Publication
(10) Pub. No.: US 2014/0003998 A1
(43) Pub. Date: Jan. 2, 2014

(54) Plasma Sterilization System

(71) Applicants: Mark A. Franklin, Scottsdale, AZ (US); William J. Franklin, Phoenix, AZ (US)

(72) Inventors: Mark A. Franklin, Scottsdale, AZ (US); William J. Franklin, Phoenix, AZ (US)

(21) Appl. No.: 13/720,545

(22) Filed: Dec. 19, 2012

Related U.S. Application Data
(60) Provisional application No. 61/582,167, filed on Dec. 30, 2011.
(60) Provisional application No. 61/638,977, filed on Apr. 26, 2012.

(51) Int. Cl. A61L 2/14

(52) U.S. Cl. CPC A61L 2/14
USPC 422/29; 422/186.29

(57) ABSTRACT

An improved system relating to sterilizing the surfaces of objects using a dual-frequency plasma-based process.

### CROSS-REFERENCE TO RELATED APPLICATION

The present application is related to and claims priority from prior provisional application Ser. No. 61/638,977, filed Apr. 26, 2012, entitled “PLASMA STERILIZATION SYSTEMS”; and, this application is related to and claims priority from prior provisional application Ser. No. 61/582,167, filed Dec. 30, 2011, entitled “PLASMA STERILIZATION SYSTEMS”, the contents of all of which are incorporated herein by this reference and are not admitted to be prior art with respect to the present invention by the mention in this cross-reference section.

### BACKGROUND

This invention relates to providing improved plasma sterilization systems. More particularly, this invention relates to providing an improved system for sterilizing the surfaces of items using high-density plasma with low ion-bombardment.

Sterilization is an essential component in many medical processes and procedures. Such sterilization processes involve the elimination of microbial life and similar disease-causing agents. It has been reported that nearly half of all medical products are currently made of plastics. These products consist of disposable tools, implants, etc. Materials such as Ultra-high-molecular-weight polyethylene (UHMWPE), polyvinyl chloride (PVC), or polyethylene terephthalate (PET) are heat-sensitive and cannot be readily sterilized using traditional autoclave processes. Traditional chemical sterilization of such items can also prove challenging. Improving the performance, efficiency, and cost of such sterilization processes would be of benefit to many within, and served by, the medical field.

### Objects and Features of the Invention

A primary object and feature of the present invention is to provide a system addressing the above-mentioned need(s). It is a further object and feature of the present invention to provide such a system for sterilizing the surfaces of three-dimensional objects using a plasma-based process. It is another object and feature of the present invention to provide such a system utilizing a high ion-density source while producing low to zero ion exposure at the treated surfaces. It is a further object and feature of the present invention to provide such a system wherein such suppression of ion exposure permits sterilization to be administered without appreciable harm to target surfaces and further enables sterilization to occur at relatively low temperatures, preferably less than about 50 degrees Celsius. It is a further object and feature of the present invention to provide such a system having a source that generates high radical concentrations at the surfaces of the treated item due to the elimination of radical displacement by ion bombardment.

A further primary object and feature of the present invention is to provide such a system that is efficient, inexpensive, and useful. Other objects and features of this invention will become apparent with reference to the following descriptions.

### SUMMARY OF THE INVENTION

In accordance with a preferred embodiment hereof, this invention provides a system relating to biological sterilization of at least one item comprising: at least one plasma generator configured to generate sterilizing plasma comprised of sterilization constituents capable of such biological sterilization and surface-harming constituents capable of harming surfaces of the at least one item to be sterilized; at least one interactor configured to promote interaction between the sterilizing plasma and the at least one item to be sterilized; and at least one electromagnetic filter structured and arranged to electromagnetically filter the surface-harming constituents from the sterilization constituents; wherein such at least one electromagnetic filter selectively passes, to the surfaces of the at least one item, a portion of such sterilization constituents while suppressing passage, to the surfaces of the at least one item, a portion of such surface-harming constituents; wherein the at least one item is sterilized by exposure to such sterilization constituents capable of biological-sterilization; and wherein surface harm to the at least one item is reduced by suppressing passage of the surface-harming constituents to the surfaces of the at least one item.

Moreover, it provides such a system wherein: such at least one plasma generator comprises at least one first radio-frequency generator configured to generate at least one first plasma-inducing radio-frequency signal; such at least one electromagnetic filter comprises at least one second radio-frequency generator configured to generate at least one second radio-frequency signal assisting such selective filtering; such at least one second radio-frequency signal generated by such at least one second radio-frequency generator comprises a lower frequency than such at least one first plasma-inducing radio-frequency signal; and such at least one second radio-frequency generator is configured to generate such at least one second radio-frequency signal at a frequency that is a non-integral multiple of such at least one first plasma-inducing radio-frequency signal.

Additionally, it provides such a system wherein such at least one interactor comprises at least one enclosure to enclose an active plasma environment containing, in interactive proximity, the sterilizing plasma and the at least one item to be sterilized. Also, it provides such a system wherein such at least one plasma generator is structured and arranged to maintain the active plasma environment within such at least one enclosure at a temperature less than about 50 degrees Celsius. In addition, it provides such a system further comprising: at least one antenna emitter configured to assist emission of the at least one first plasma-inducing radio-frequency signal within such at least one enclosure; wherein such at least one antenna emitter is operably coupled with such at least one first radio-frequency generator; and wherein such at least one antenna emitter comprises at least one dielectric coating. And, it provides such a system further comprising: at least one electrode emitter configured to assist emission of the at least one second radio-frequency signal within such at least one enclosure; and at least one support to support, within such at least one enclosure, the at least one item during such sterilization; wherein such at least one support is located between such at least one antenna emitter and such at least one electrode emitter.

Further, it provides such a system wherein: such sterilization constituents capable of biological-sterilization at least include radical constituents and ultraviolet photon constituents; such surface-harming constituents capable of surface harm at least include charged ion constituents; and such at least one second radio-frequency generator and such at least one electrode emitter are configured to produce at least one interaction between such at least one second radio-frequency signal and such the sterilizing plasma suppressing interaction of the charged ion constituents with the at least one item to be sterilized. Even further, it provides such a system wherein such first radio-frequency generator is configured to produce the at least one first plasma-inducing radio-frequency signal at a frequency of about 2.45 GHz. Moreover, it provides such a system wherein such at least one second radio-frequency generator produces such at least one second radio-frequency signal at a frequency of about 399 KHz.

Additionally, it provides such a system wherein such at least one antenna array is configured electrically to be at or near ground potential. Also, it provides such a system wherein: such at least one second radio-frequency generator utilizes at least one radio-frequency match network structured and arranged to match an output load of such at least one second radio-frequency generator to dynamic impedances of the active plasma environment contained within such at least one enclosure; and such at least one electrode emitter is electrically coupled with such at least one second radio-frequency generator through such at least one radio-frequency match network.

In addition, it provides such a system further comprising at least one electrode-spacing adjuster to assist adjusting the spacing between such at least one electrode and such at least one antennae array. And, it provides such a system wherein such at least one electrode-spacing adjuster is further configured to assist adjusting the spacing between such at least one support and such at least one antennae array.

Further, it provides such a system further comprising at least one a vacuum pump operatively connected to such at least one enclosure. Even further, it provides such a system further comprising at least one programmable coordinating controller structured and arranged to provide programmed coordination and control of the operation of such at least one plasma generator, such at least one interactor, and such at least one electromagnetic filter.

In accordance with another preferred embodiment hereof, this invention provides a method relating to biological sterilization of at least one item comprising the steps of: providing at least one plasma generator configured to generate sterilizing plasma comprised of sterilization constituents capable of such biological sterilization and surface-harming constituents capable of harming surfaces of the at least one item to be sterilized; providing at least one interactor configured to promote interaction between the sterilizing plasma and the at least one item to be sterilized; providing at least one electromagnetic filter structured and arranged to electromagnetically filter the surface-harming constituents from the sterilization constituents; and configuring such at least one electromagnetic filter to selectively pass, to the surfaces of the at least one item, a portion of such sterilization constituents while suppressing passage, to the surfaces of the at least one item, a portion of such surface-harming constituents; wherein the at least one item is sterilized by exposure to such sterilization constituents capable of biological-sterilization; and wherein surface harm to the at least one item is reduced by suppressing passage of the surface-harming constituents to the surfaces of the at least one item.

Moreover, it provides such a method further comprising the step of configuring such at least one interactor to comprise at least one enclosure configured to enclose an active plasma environment containing, in interactive proximity, the sterilizing plasma and the at least one item to be sterilized. Additionally, it provides such a method further comprising the steps of: introducing the at least one item to be sterilized into such at least one enclosure; and generating such sterilizing plasma within such at least one enclosure; wherein such at least one electromagnetic filter electromagnetically filters the surface-harming constituents from the sterilization constituents; wherein such at least one electromagnetic filter selectively passes, to the surfaces of the at least one item, a portion of such sterilization constituents while suppressing passage, to the surfaces of the at least one item, a portion of such surface-harming constituents; wherein the at least one item is sterilized by exposure to such sterilization constituents capable of biological-sterilization; and wherein surface harm to the at least one item is reduced by suppressing passage of the surface-harming constituents to the surfaces of the at least one item. Also, it provides such a method further comprising the step of: configuring such at least one plasma generator to comprise at least one first radio-frequency generator configured to generate at least one first plasma-inducing radio-frequency signal; configuring such at least one electromagnetic filter to comprise at least one second radio-frequency generator configured to generate at least one second radio-frequency signal assisting such selective filtering; wherein such at least one second radio-frequency signal generated by such at least one second radio-frequency generator comprises a frequency lower than such at least one first plasma-inducing radio-frequency signal; and wherein such at least one second radio-frequency generator is configured to generate such at least one second radio-frequency signal at a frequency that is a non-integral multiple of such at least one first plasma-inducing radio-frequency signal. In addition, it provides such a method further comprising the step of configuring such at least one plasma generator to maintain the active plasma environment within such at least one enclosure at a temperature less than about 50 degrees Celsius. And, it provides such a method further comprising step of providing at least one electrode-spacing adjuster to assist adjusting the spacing between at least one electrode emitting such at least one second radio-frequency signal and at least one antennae array emitting such at least one first plasma-inducing radio-frequency signal.

Further, it provides such a method further comprising the step of configuring such at least one electrode-spacing adjuster to assist adjusting the spacing between such at least one item and such at least one antennae array. Even further, it provides such a method comprising the step of providing at least one programmable coordinating controller structured and arranged to provide programmed coordination and control of the operation of such at least one plasma generator, such at least one interactor, and such at least one electromagnetic filter.

In accordance with another preferred embodiment hereof, this invention provides a system relating to biological sterilization of at least one medical item comprising: plasma generator means for generating sterilizing plasma comprised of sterilization constituents capable of such biological sterilization and surface-harming constituents capable of harming surfaces of the at least one medical item to be sterilized; interactor means for promoting interaction between the sterilizing plasma and the at least one medical item to be sterilized; and electromagnetic filter means for electromagnetically filtering the surface-harming constituents from the sterilization constituents; wherein such electromagnetic filter means selectively passes, to the surfaces of the at least one medical item, a portion of such sterilization constituents while suppressing passage, to the surfaces of the at least one item, a portion of such surface-harming constituents; wherein the at least one medical item is sterilized by exposure to such sterilization constituents capable of biological-sterilization; and wherein surface harm to the at least one medical item is reduced by suppressing passage of the surface-harming constituents to the surfaces of the at least one medical item.

In accordance with preferred embodiments hereof, this invention provides each and every novel feature, element, combination, step and/or method disclosed or suggested by this patent application.

### Definitions and Supporting Data

Plasma: Plasma is a distinct state of matter similar to a gas in which a portion of the particles are ionized. Except near the electrodes, where sheaths are developed containing very few electrons, the partially ionized gas generally contains an equal number of positive and negative charges (as well as a number of non-ionized gas particles) so that the resultant charge is substantially neutral. In general, plasma is electrically conductive so that it responds strongly to electromagnetic fields.

Sterilization: Sterilization is an act or process, physical or chemical that destroys or eliminates all forms of life, especially microorganisms. Conventional sterilization techniques, such as those using autoclaves, ovens, and chemicals like ethylene oxide (EtO), rely on irreversible metabolic inactivation or on breakdown of vital structural components of the microorganism.

Etching: Etching in a discharge environment is the result of active species reacting with a substrate to form volatile products. In plasma etching, the reactive species generally comprise ions and activated neutrals.

### BRIEF DESCRIPTION OF THE DRAWINGS

FIG. 1 is a schematic diagram, illustrating preferred arrangements of a plasma-based surface sterilizer, according to preferred embodiments of the present invention.

FIG. 2 is schematic diagram, showing sterilizing constituents of sterilizing plasma utilized by the preferred embodiments of the present invention.

FIG. 3 shows a flow diagram, illustrating a plasma-based surface sterilization process, according to a preferred method of the present invention.

### DETAILED DESCRIPTION OF THE BEST MODES AND PREFERRED EMBODIMENTS OF THE INVENTION

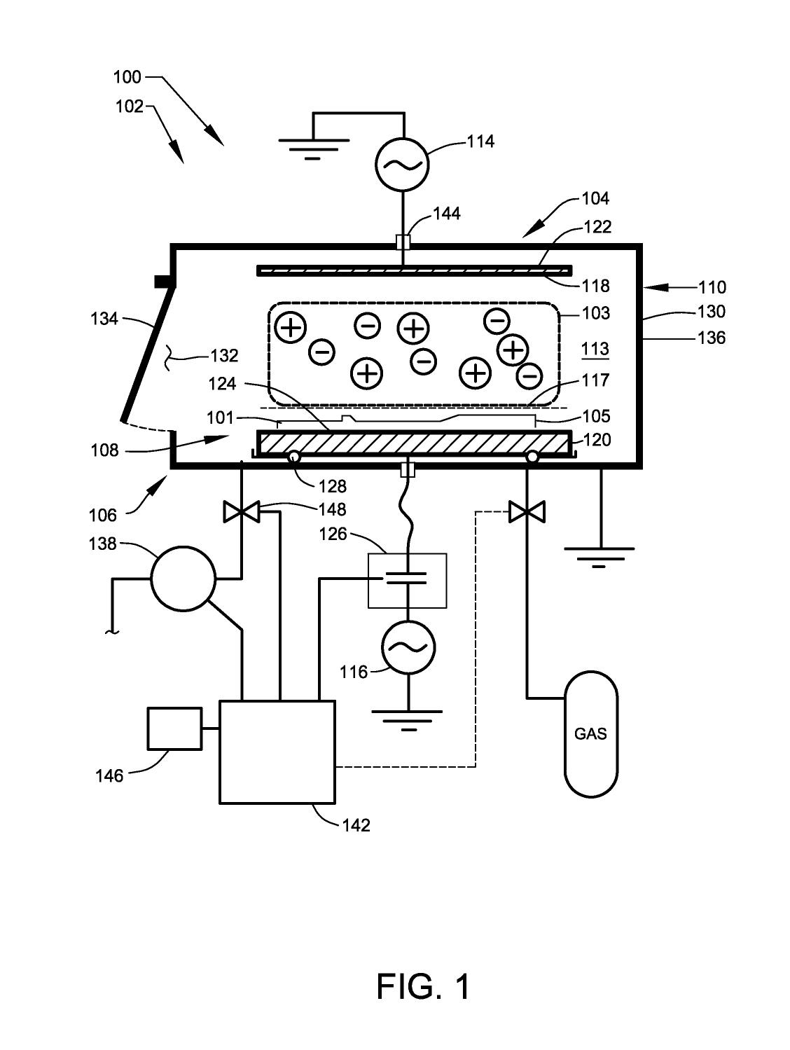

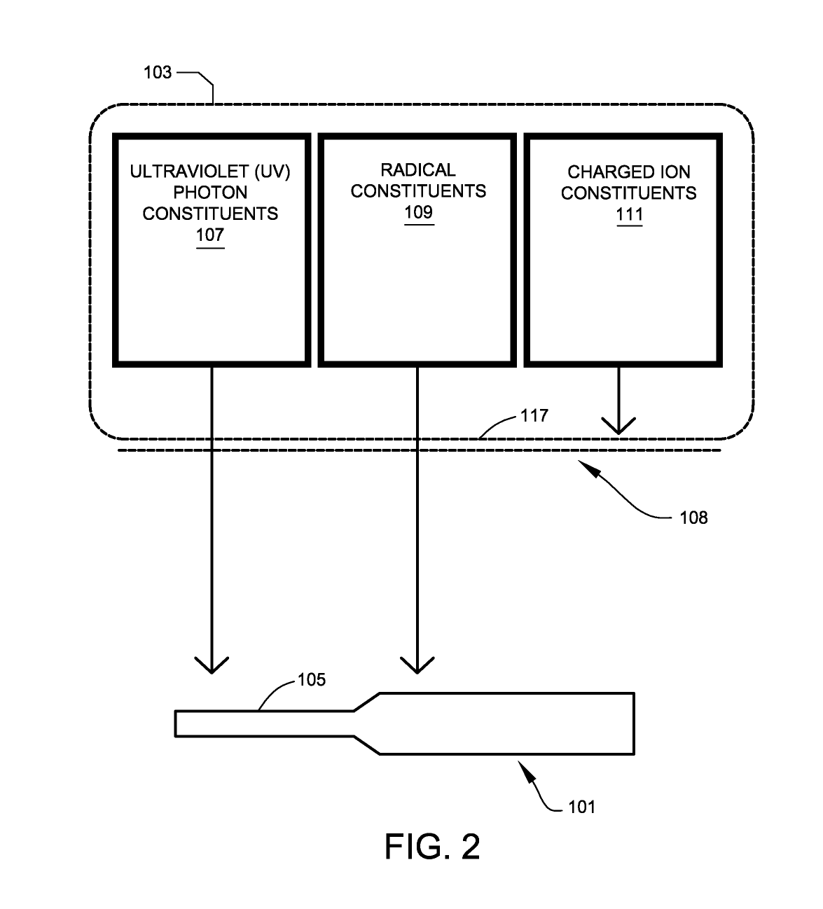

FIG. 1 shows a schematic diagram illustrating plasma-based surface sterilizer 102 according to a preferred embodiment of plasma sterilization system 100. FIG. 2 is schematic diagram showing sterilizing constituents of sterilizing plasma utilized by the preferred embodiments of the present invention.

Preferred embodiments of plasma sterilization system 100 are preferably configured to biologically sterilize of one or more three-dimensional target items 101 by exposing the items to select constituents of sterilizing plasma 103. The plasma sterilization process provided by the present system preferably utilizes a plasma source of high ion-density, which is preferably modified electromagnetically to produce low to zero ion exposure at the treated surfaces of the item. Such suppression of ion exposure permits sterilization to be administered without appreciable harm to target surfaces and further enables sterilization to occur at relatively low temperatures, preferably less than about 50 degrees Celsius. Thus, the present system allows for biological sterilization of heat sensitive items, such as, for example, polymer-based medical instruments, which often cannot be subjected to the temperatures associated with conventional autoclaves and ovens.

Plasma sterilization employed by the present system generally operates by passing specific constituents of sterilizing plasma 103 to the surfaces 105 of target items 101 while suppressing the passage of others. Preferred sterilizing constituents of sterilizing plasma 103 passed to items 101 include ultraviolet (UV) photon constituents 107 and radical constituents 109, as illustrated diagrammatically in FIG. 2.

The general mechanisms of plasma sterilization implemented by the present system include destruction by UV irradiation of the genetic material of a microorganism, erosion of the microorganism, atom by atom, through intrinsic photo-desorption, and erosion of the microorganism, atom by atom, through surface interaction. UV irradiation is a statistical process requiring sufficient damage of the DNA strands of the microorganism. Photon-induced desorption results from UV photons breaking chemical bonds in the microorganism material. Erosion of the microorganism by surface interaction results from the adsorption by the microorganism of reactive species from the plasma (glow or afterglow), which subsequently undergo chemical reactions to form volatile compounds. The reactive species can be atomic and molecular radicals, for example, O and O₃, respectively, and excited molecules in a meta-stable state, for example, the ¹O₂ singlet state, as generally illustrated in the diagram of FIG. 2. It is surmised that the surface interaction mechanism is further enhanced by the presence of UV photon constituents 107.

Constituents of sterilizing plasma 103 preferably suppressed from passage to items 101 by the system preferably comprise energetic constituents capable of harming surfaces of the item during the sterilization process. Such surface-harming constituents include charged ion constituents 111 capable of producing surface etching, surface erosion, and/or other unwanted surface modification. In the present disclosure, “unwanted surface modification” shall be generally defined as any chemical or mechanical change to the surface properties of the item that significantly reduce the functionality or durability the treated item. Such “surface modification” may include altering adhesion properties, wetting/hydrophilicity properties, surface hardness, resiliency, oxidation, biomedical compatibility, etc. In the present disclosure, the terms “etching” and “erosion” shall be generally defined as the damaging ejection of atoms and molecules from the surface of the item by ion bombardment. It is essential to any commercially-viable sterilization system that such surface harm be avoided during the sterilization process. The preferred ion-controlling mechanism employed by preferred embodiments of plasma sterilization system 100 is the establishment of an ion-impervious sheath region located between the bulk sterilizing plasma 103 and item 101.

Referring again to the diagram of FIG. 1, plasma-based sterilizer 102 preferably comprises three principal subsystems generally identified herein as plasma generator 104, interactor 106, and electromagnetic filter 108, as shown. Plasma generator 104 is preferably configured to generate sterilizing plasma 103, preferably utilizing radio frequency (RF) energy (at least embodying herein plasma generator means for generating sterilizing plasma comprised of sterilization constituents capable of such biological sterilization and surface-harming constituents capable of harming surfaces of the at least one medical item to be sterilized). As previously noted, the bulk sterilizing plasma 103 generated by plasma generator 104 at least comprises UV photon constituents 107, radical constituents 109, and ion constituents 111. Generation of high-density plasma using RF signals is well known in the art and will therefore not be discussed in detail.

In general terms, interactor 106 preferably comprises one or more structures functioning to promote interaction between sterilizing plasma 103 and item 101 to be sterilized (at least embodying herein interactor means for promoting interaction between the sterilizing plasma and the at least one medical item to be sterilized). In terms of specific preferred embodiments of the system, interactor 106 preferably comprises an enclosable processing chamber 110, which is preferably designed to hold the active plasma environment 113 and item 101 in interactive proximity during the sterilization cycle (at least embodying herein at least one enclosure to enclose an active plasma environment).

Electromagnetic filter 108 is preferably structured and arranged to selectively filter surface-harming ion constituents 111 from the UV photon constituents 107 and radical constituents 109 (i.e., the sterilization constituents) present in the active plasma environment 113. More specifically, electromagnetic filter 108 selectively passes, to surfaces 105 of item 101, a portion of UV photon constituents 107 and radical constituents 109 of the bulk sterilizing plasma 103 while suppressing passage, to surfaces 105 of item 101, a substantial portion of such ion constituents 111.

Electromagnetic filter 108 is preferably enabled by the establishment of an ion-impervious sheath region located between the bulk sterilizing plasma 103 and item 101 (at least embodying herein electromagnetic filter means for electromagnetically filtering the surface-harming constituents from the sterilization constituents). This ion-impervious boundary region of the bulk plasma, identified herein as plasma sheath 117, is located adjacent the bottom electrode 120 on which item 101 is supported.

The preferred ion-shielding properties of plasma sheath 117 are preferably modulated by the introduction of a second RF signal into processing chamber 110. Charged ion species generated by the first RF signal that are out of phase with the second RF signal are prevented from passing through plasma sheath 117 to interact with item 101. In practice, surfaces 105 of item 101 may be fully shielded from bombardment by ion constituents 111 by optimized implementations of the presently-disclosed process. Thus, a target item 101 may be sterilized by exposure to only the sterilization constituents of the plasma while surface harm to item 101 is reduced or eliminated, preferably by suppressing bombardment of item 101 by ion constituents 111.

In specific reference to the diagram of FIG. 1, plasma-based sterilizer 102 preferably comprise two radio frequency (RF) generators identified herein as first radio frequency (RF) generator 114, second RF generator 116. Processing chamber 110 preferably encloses an upper antenna emitter 118, a lower isolated electrode 120, and at least one support 124 structured and arranged to support items 101, as shown.

Preferably, antenna emitter 118 is operably coupled with first radio-frequency generator 114, as shown, and is preferably configured to assist the radiation of the first plasma-inducing radio-frequency signal within processing chamber 110. Antenna emitter 118 is preferably configured to be at ground potential and preferably comprises a dielectric coating 122. During the plasma sterilization cycle, the first plasma-inducing radio-frequency signal radiated by antenna emitter 118 preferably functions to form sterilizing plasma 103 by energizing gas within processing chamber 110. In the present preferred embodiment, oxygen gas inside processing chamber 110 is converted to sterilizing plasma 103. The power settings of first RF generator 114 are preferably selected to generate RF signals above those required for basic plasma ignition (that is, for any given operating gas pressure within the sterilization chamber).

Lower isolated electrode 120 is preferably configured to assist emission of the second radio-frequency signal within processing chamber 110. The lower isolated electrode 120 is preferably coupled to second RF generator 116, as shown. RF energies produced by first radio frequency (RF) generator 114 and second RF generator 116 are preferably radiated into processing chamber 110 substantially simultaneously.

Electrode 120 is preferably coupled to second RF generator 116 utilizing at least one radio-frequency (RF) match network 126, as shown. RF match network 126 is preferably configured to match an output load of the RF generator(s) to the dynamic impedances of active plasma environment 113 within processing chamber 110. RF match network 126 preferably ensures that the capacitive impedance that exists between the upper and lower electrodes is controlled and any RF energy reflection to the input circuitry is attenuated. Preferably, the matching circuitry used to couple the RF power to the chamber is derived from configurations well-known to those of ordinary skill in the art of plasma-based processes. Upon reading this specification, those with ordinary skill in the art will now appreciate that, under appropriate circumstances, considering such issues as design preference, intended use, marketing preferences, cost, technological advances, etc., other match network arrangements such as, for example, utilizing a match network to coordinate overall operation of both RF sources, etc., may suffice.

Lower isolated electrode 120 is preferably configured to comprise support 124, as shown, or alternately preferably, is located proximally adjacent support 124. Support 124 is preferably configured to support item 101 within processing chamber 110 during the sterilization process, preferably at an optimized position relative to the upper and lower electrodes. Support 124 is preferably configured to place item 101 substantially between antenna emitter 118 and electrode 120, as shown.

Preferably, the spacing between the electrodes is adjustable to optimize the chamber configuration for specific sterilization parameters. More particularly, plasma-based sterilizer 102 preferably comprises at least one electrode-spacing adjuster 128 to assist adjusting the spacing between the lower isolated electrode 120 and antenna emitter 118. Most preferably, the lower isolated electrode 120 and associated support 124 are preferably configured to be movable to permit the spacing between the lower isolated electrode 120 and antenna emitter 118 to be adjusted.

Second RF generator 116 and the isolated electrode 120 preferably interoperate to modulate the ion-shielding properties at plasma sheath 117 to produce filter-like ion-exclusion properties at the boundary of the bulk sterilizing plasma 103. In particular, the generated second radio-frequency signal preferably functions to electromagnetically modulate the lower plasma sheath 117 to prevent the charged ion constituents 111 of sterilizing plasma 103 from penetrating through the sheath boundary. Charged ion species generated by the first RF signal that are out of phase with the second RF signal are constrained from passing through plasma sheath 117 by the applied voltage. The transitional region of plasma sheath 117 is preferably modulated to comprise a non-neutral field potential that effectively retards rather than promotes ion acceleration across the boundary to the substrate.

The power settings of first RF generator 114 are preferably adjusted to produce sterilizing plasma 103 having high ion density and above average radical (particle) generation. Radical constituents 109, which provide one of the two fundamental modes of sterilization, have a relatively short life (lasting about 0.1 second) and must therefore be constantly be generated. It is thus important to generate a large number of radicals during the plasma cycle. The selected RF signal generated by first RF generator 114 therefore comprises a high frequency profile. More specifically, the RF signal generated by first RF generator 114 preferably falls within at least one microwave-frequency range, preferably a microwave frequency located within the 2.400 GHz to 2.500 GHz of the Industrial Scientific Medical (ISM) band.

Second radio-frequency signal is preferably applied to the substrate at an atypically low frequency relative to the first RF signal and falls outside the typical ISM spectrum. It was determined that the use of such a non-standard wide frequency differential yielded unexpectedly high ion shielding behaviors at the region of the sheath boundary. It is also noted that when the selected second RF voltage is applied to the lower electrode, the ion bombardment energy at the target is modified without affecting significantly the density of the bulk sterilizing plasma 103.

The lower frequency of second radio-frequency signal is preferably selected to tune plasma environment 113 to produce both effective ion shielding and maximum exposure levels of UV photon constituents 107 and radical constituents 109 at item 101. It is noted that such ion filtering eliminates heating associated with ion bombardment; thus, the sterilization process provided by the present system occurs at relatively low temperatures, preferably less than or equal to about 50-degrees Celsius. This preferred feature of the present system enables preservation of the integrity of polymer-based items, such as, medical instruments. Heat sensitive materials like UHMWPE, PVC or PS can thus be treated without damage (the melting point of UHMWPE is about 135-degrees Celsius; however, the mean temperature of the material should be maintained lower than 80-degrees Celsius to preserve mechanical integrity).

In one preferred embodiment of plasma-based sterilizer 102, first RF generator 114 is preferably configured to produce the first plasma-inducing radio-frequency signal at an average frequency of about 2.45 GHz (comprising a wavelength of about 122 millimeters). This preferred frequency produces plasma of sufficient density and electron temperatures required for sterilization.

Second RF generator 116 preferably produces the second radio-frequency signal at an average frequency of about 400 KHz (more preferably about 399 khZ). It should again be noted that the preferred frequency relationship between the signals produced by first RF generator 114 and second RF generator 116 are those of non-integral multiples. Such preferred use of non-commensurate frequencies avoids RF resonance and similar dynamics as the preferred plasma environment is established.

Upon reading this specification, those with ordinary skill in the art will now appreciate that, under appropriate circumstances, considering such issues as design preference, intended use, cost, structural requirements, available materials, technological advances, etc., other frequency combinations such as, for example, higher radio frequencies, lower radio frequencies, alternate non-integral combinations, etc., may suffice.

As diagrammatically illustrated in FIG. 1, plasma-based sterilizer 102 preferably comprises additional sub-components necessary to implement the above described sterilization process. For example, processing chamber 110 preferably comprises an outer gas-tight wall 130 having at least one access port 132 capable of passing item 101 or items 101 therethrough. Access port 132 is preferably sealed by at least one chamber door 134 that is preferably capable of achieving a pressure seal surrounding access port 132, thus enabling the preferred formation of an enclosed pressure boundary 136 surrounding item 101 during the plasma sterilization cycle. Preferably, interior chamber components can be rounded and finished to prevent arcing during plasma cycling. The RF signal generated by first radio frequency (RF) generator 114 is preferably coupled to antenna emitter 118 by means of RF electrical feed through 144 (or waveguide) passing through the outer gas-tight chamber wall 130. RF electrical feed through 144 preferably includes at least one pressure seal to ensure a vacuum seal where the assembly passes through chamber wall 130. Preferably, chamber wall 130 is preferably configured to comprise an electrical ground potential, as shown.

After placement of item 101 or items 101 on or within support 124, gas within processing chamber 110 is partially evacuated by at one pressure regulating circuit, preferably comprising vacuum pump 138. Vacuum pump 138 may preferably comprise a conventional dry mechanical pump. Preferably, vacuum pump 138 is selectively brought into fluidic communication with processing chamber 110 by at least one valve 148 under the control of controller 142. Once a sufficient reduced pressure level has been achieved, upper antenna emitter 118 and lower isolated electrode 120 are preferably energized, preferably substantially contemporaneously, by their respective RF generators. As a preferred example, power is applied at a low pressure (between about 0.5 and 10 Torr) permitting the gas within processing chamber 110 to be ignited by the RF energy generated by first RF generator 114 to produce charged ions, UV photons, and radical constituents. Upon reading this specification, those with ordinary skill in the art will now appreciate that, under appropriate circumstances, considering such issues as sterilization duration, composition of the items to be sterilized, technological advances, etc., other component arrangements such as, for example, providing a means for introducing a feed-gas mixture into the processing chamber during the plasma sterilization cycle, etc., may suffice.

Preferred embodiments of plasma-based sterilizer 102 preferably comprise at least one programmable controller 142 structured and arranged to provide programmed coordination and control of the operation of plasma-based sterilizer 102 (at least embodying herein at least one programmable coordinating controller structured and arranged to provide programmed coordination and control of the operation of such at least one plasma generator, such at least one interactor, and such at least one electromagnetic filter). Controller 142 preferably functions to monitor apparatus operation and pass computer programmed and real-time commands to and from the mechanical systems of plasma-based sterilizer 102. This preferred arrangement has the advantage of enabling pre-programmed automation and automated per-cycle tuning of the plasma sterilization process. Controller 142 is preferably implemented using a general purpose computer; for example, a PC-based computer running dedicated software. Alternatively preferably, controller 142 is preferably implemented within micro-controller architecture or a combination of timers and relays, which are preferably arranged to provide logic and control functions. Preferred embodiments of plasma-based sterilizer 102 preferably comprise at least one user interface 146 to permit user inputs to controller 142 and to provide data concerning system operations.

Controller 142 preferably receives and processes sensor data and similar feedback relating to the operation of plasma-based sterilizer 102. Such sensor data may preferably include pressure sensors, voltage and current sensors, sensors to measure charged-particle densities, sensors to measure temperatures within plasma environment 113, etc. The plasma measurements are preferably used to monitor the condition of plasma environment 113 or to furnished data to controller 142 for use in controlling the plasma sterilization process. Upon reading this specification, those with ordinary skill in the art will now appreciate that, under appropriate circumstances, considering such issues as design preference, user preferences, intended use, cost, structural requirements, available materials, technological advances, etc., the inclusion of other system components such as, for example, feed-gas valves, mass flow controllers, power distribution devices, automated item handling features, etc., may suffice.

A surface exposed to plasma sterilization, according to the present system, preferably produces surface decomposition (erosion) of microorganisms located on or adjacent the target surfaces. Such interaction preferably produces surface decomposition of the microorganism, essentially atom by atom. In more specific terms, the decomposition of the microorganism is a result of the adsorption, by the target microorganisms, of reactive radicals from the plasma. As a result, the microorganisms subsequently undergo chemical reactions to form volatile compounds that results in spontaneous surface decomposition.

As illustrated in the diagram of FIG. 2, the reactive radicals generated by the process can be atomic and molecular radicals, for example, O and O₃, respectively, and excited molecules in a meta-stable state. This chemistry, which occurs under thermodynamic equilibrium conditions, yields small molecules (e.g., CO₂, H₂O) that are the final products of the oxidation process. In this case, the sterilization mechanism is enhanced by UV photons (UV-induced etching), the photons acting synergistically with the reactive species, thereby accelerating the elimination rate of microorganisms. This UV-induced chemistry, which occurs under non-equilibrium conditions, can result in the preferred desorption of radicals and molecules, at both the intermediate and final stages of oxidation of the microorganism.

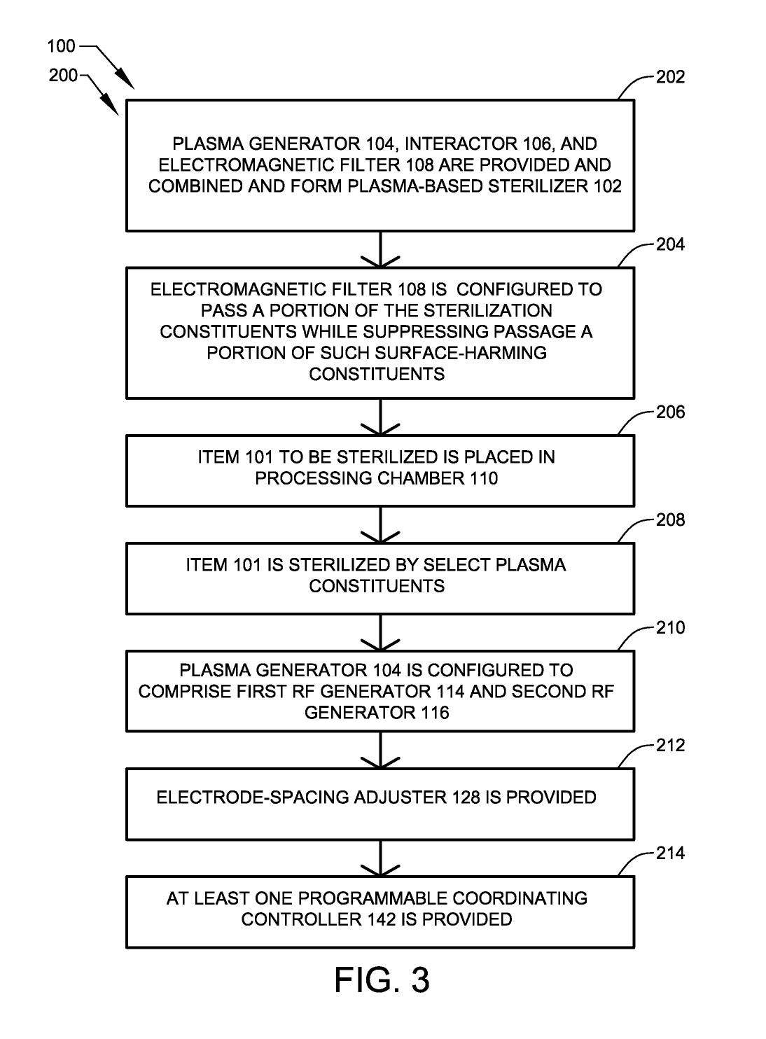

FIG. 3 shows a flow diagram illustrating the preferred steps of plasma-based surface sterilization process 200 according to a preferred method of the present invention. Plasma-based surface sterilization process 200 preferably comprises a method enabling biological sterilization, which is generally embodied in the following steps.

In initial step 202 of plasma-based surface sterilization process 200, plasma generator 104 (at least comprising first RF generator 114), interactor 106 (at least in the form of processing chamber 110), and electromagnetic filter 108 (at least comprising second RF generator 116) are provided and combined and form plasma-based sterilizer 102. In subsequent step 204 of plasma-based surface sterilization process 200, electromagnetic filter 108 is preferably configured to selectively pass, to the surfaces of item 101, a portion of the sterilization constituents of sterilizing plasma 103 while suppressing passage, to the surfaces of item 101, a portion of such surface-harming constituents.

Next, as indicated by preferred step 206, at least on item 101 to be sterilized is placed in processing chamber 110. Next, as indicated in preferred step 208, at least one sterilizing plasma 103 is preferably generated within processing chamber 110 containing item 101. Within step 208, item 101 is preferably exposed to sterilization constituents capable of biological-sterilization at least including radical constituents and ultraviolet photon constituents. Within step 208, surface-harming constituents are suppressed from interaction with item 101 by electromagnetic filter 108. In this preferred manner, item 101 is sterilized by exposure to such sterilization constituents capable of biological-sterilization and surface harm to item 101 is reduced by suppressing passage of the surface-harming constituents to surfaces 105 of item 101.

In preferred step 210, plasma generator 104 is preferably configured to comprise first RF generator 114 and second RF generator 116. As previously described, the second radio-frequency signal generated by second RF generator 116 preferably comprises a lower frequency than the first plasma-inducing radio-frequency signal. As noted previously, second RF generator 116 is preferably configured to generate the second radio-frequency signal at a frequency that is a non-integral multiple of the first plasma-inducing radio-frequency signal. Implementation of step 210 preferably includes configuring plasma generator 104 to maintain active plasma environment 113 at a temperature less than about 50-degrees Celsius.

In preferred step 212, at least one electrode-spacing adjuster 128 is provided to assist adjusting the spacing between electrode 120 and antenna emitter 118. Preferably, electrode-spacing adjuster 128 assists adjusting the spacing between support 124 and antenna emitter 118. Next, as indicated in preferred step 214, at least one programmable coordinating controller 142 is provided to enable provide programmed coordination and control of the operation of plasma generator 104, interactor 106, and electromagnetic filter 108.

### System Advantages

The preferred source embodiments, apparatus arrangements, and methods of the present system at least provide the following advantages:
a) high ion density production;
b) low to zero ion exposure to treated surfaces of item 101 (treated surfaces are preferably located in the glow discharge increasing UV photon exposure); and
c) production of high radical concentrations at the treated surface due to the elimination of radical displacement by ion bombardment.

Although applicant has described applicant's preferred embodiments of this invention, it will be understood that the broadest scope of this invention includes modifications such as diverse shapes, sizes, and materials. Such scope is limited only by the below claims as read in connection with the above specification. Further, many other advantages of applicant's invention will be apparent to those skilled in the art from the above descriptions and the below claims.

What is claimed is:

1) A system relating to biological sterilization of at least one item comprising:
a) at least one plasma generator configured to generate sterilizing plasma comprising sterilization constituents capable of the biological sterilization and surface-harming constituents capable of harming surfaces of the at least one item to be sterilized;
b) at least one interactor structured and arranged to promote interaction between such sterilizing plasma and the at least one item to be sterilized; and
c) at least one electromagnetic filter structured and arranged to electromagnetically filter such surface-harming constituents from such sterilization constituents;
d) wherein said at least one electromagnetic filter selectively passes, to surfaces of the at least one item, a portion of such sterilization constituents while suppressing passage, to the surfaces of the at least one item, a portion of such surface-harming constituents;
e) wherein the at least one item is sterilized by exposure to such sterilization constituents capable of biological-sterilization; and
f) wherein surface harm to the at least one item is reduced by suppressing passage of such surface-harming constituents to the surfaces of the at least one item.

2) The system, according to claim 1, wherein:
a) said at least one plasma generator comprises at least one first radio-frequency generator structured and arranged to generate at least one first plasma-inducing radio-frequency signal;
b) said at least one electromagnetic filter comprises at least one second radio-frequency generator structured and arranged to generate at least one second radio-frequency signal assisting such selective filtering;
c) such at least one second radio-frequency signal generated by said at least one second radio-frequency generator comprises a lower frequency than such at least one first plasma-inducing radio-frequency signal; and
d) said at least one second radio-frequency generator is configured to generate such at least one second radio-frequency signal at a frequency that is a non-integral multiple of such at least one first plasma-inducing radio-frequency signal.

3) The system, according to claim 2, wherein said at least one interactor comprises at least one enclosure to enclose an active plasma environment containing, in interactive proximity, such sterilizing plasma and the at least one item to be sterilized.

4) The system, according to claim 3, wherein said at least one plasma generator is structured and arranged to maintain such active plasma environment within said at least one enclosure at a temperature less than about 50 degrees Celsius.

5) The system, according to claim 3, further comprising:
a) at least one antenna emitter structured and arranged to assist emission of such at least one first plasma-inducing radio-frequency signal within said at least one enclosure;
b) wherein said at least one antenna emitter is operably coupled with said at least one first radio-frequency generator; and
c) wherein said at least one antenna emitter comprises at least one dielectric coating.

6) The system, according to claim 5, further comprising:
a) at least one electrode emitter configured to assist emission of such at least one second radio-frequency signal within said at least one enclosure; and
b) at least one support to support, within said at least one enclosure, the at least one item during such sterilization;
c) wherein said at least one support is located between said at least one antenna emitter and said at least one electrode emitter.

7) The system, according to claim 6, wherein:
a) such sterilization constituents capable of the biological-sterilization at least include radical constituents and ultraviolet photon constituents;
b) such surface-harming constituents capable of surface harm at least include charged ion constituents; and
c) said at least one second radio-frequency generator and said at least one electrode emitter are configured to produce at least one interaction between such at least one second radio-frequency signal and such sterilizing plasma suppressing interaction of the charged ion constituents with the at least one item to be sterilized.

8) The system, according to claim 7, wherein said first radio-frequency generator is structured and arranged to produce such at least one first plasma-inducing radio-frequency signal at a frequency of about 2.45 GHz.

9) system, according to claim 8, wherein said at least one second radio-frequency generator produces such at least one second radio-frequency signal at a frequency of about 399 KHz.

10) The system, according to claim 7, wherein said at least one antenna array is structured and arranged to electrically be at or near ground potential.

11) The system, according to claim 7, wherein:
a) said at least one second radio-frequency generator utilizes at least one radio-frequency match network structured and arranged to match an output load of said at least one second radio-frequency generator to dynamic impedances of the active plasma environment contained within said at least one enclosure; and
b) said at least one electrode emitter is electrically coupled with said at least one second radio-frequency generator through said at least one radio-frequency match network.

12) The system, according to claim 7, further comprising at least one electrode-spacing adjuster structured and arranged to assist adjusting spacing between said at least one electrode and said at least one antennae array.

13) The system, according to claim 12, wherein said at least one electrode-spacing adjuster is further structured and arranged to assist adjusting the spacing between said at least one support and said at least one antennae array.

14) The system, according to claim 7, further comprising at least one a vacuum pump operatively connected to said at least one enclosure.

15) The system, according to claim 7, further comprising at least one programmable coordinating controller structured and arranged to provide programmed coordination and control of the operation of said at least one plasma generator, said at least one interactor, and said at least one electromagnetic filter.

16) A method relating to biological sterilization of at least one item comprising the steps of:
a) providing at least one plasma generator structured and arranged to generate sterilizing plasma comprising sterilization constituents capable of the biological sterilization and surface-harming constituents capable of harming surfaces of the at least one item to be sterilized;
b) providing at least one interactor structured and arranged to promote interaction between the sterilizing plasma and the at least one item to be sterilized;
c) providing at least one electromagnetic filter structured and arranged to electromagnetically filter the surface-harming constituents from the sterilization constituents; and
d) configuring such at least one electromagnetic filter to selectively pass, to surfaces of the at least one item, a portion of such sterilization constituents while suppressing passage, to the surfaces of the at least one item, a portion of such surface-harming constituents;
e) wherein the at least one item is sterilized by exposure to such sterilization constituents capable of biological-sterilization; and
f) wherein surface harm to the at least one item is reduced by suppressing passage of such surface-harming constituents to the surfaces of the at least one item.

17) The method, according to claim 16, further comprising the step of configuring such at least one interactor to comprise at least one enclosure structured and arranged to enclose an active plasma environment containing, in interactive proximity, such sterilizing plasma and the at least one item to be sterilized.

18) The method, according to claim 17, further comprising the steps of:
a) introducing the at least one item to be sterilized into such at least one enclosure; and
b) generating such sterilizing plasma within such at least one enclosure;
g) wherein such at least one electromagnetic filter electromagnetically filters such surface-harming constituents from such sterilization constituents;
h) wherein such at least one electromagnetic filter selectively passes, to the surfaces of the at least one item, a portion of such sterilization constituents while suppressing passage, to the surfaces of the at least one item, a portion of such surface-harming constituents;
i) wherein the at least one item is sterilized by exposure to such sterilization constituents capable of biological-sterilization; and
c) wherein surface harm to the at least one item is reduced by suppressing passage of the surface-harming constituents to the surfaces of the at least one item.

19) The method, according to claim 18, further comprising the step of:
d) configuring such at least one plasma generator to comprise at least one first radio-frequency generator structured and arranged to generate at least one first plasma-inducing radio-frequency signal;
e) configuring such at least one electromagnetic filter to comprise at least one second radio-frequency generator structured and arranged to generate at least one second radio-frequency signal assisting such selective filtering;
f) wherein such at least one second radio-frequency signal generated by such at least one second radio-frequency generator comprises a frequency lower than such at least one first plasma-inducing radio-frequency signal; and
g) wherein such at least one second radio-frequency generator is configured to generate such at least one second radio-frequency signal at a frequency that is a non-integral multiple of such at least one first plasma-inducing radio-frequency signal.

20) The method, according to claim 19, further comprising the step of configuring such at least one plasma generator to maintain such active plasma environment within said at least one enclosure at a temperature less than about 50 degrees Celsius.

21) The method, according to claim 19, further comprising step of providing at least one electrode-spacing adjuster to assist adjusting spacing between at least one electrode emitting such at least one second radio-frequency signal and at least one antennae array emitting such at least one first plasma-inducing radio-frequency signal.

22) The method, according to claim 21, further comprising the step of configuring such at least one electrode-spacing adjuster to assist adjusting the spacing between such at least one item and such at least one antennae array.

23) The method, according to claim 19, further comprising the step of providing at least one programmable coordinating controller structured and arranged to provide programmed coordination and control of operation of such at least one plasma generator, such at least one interactor, and such at least one electromagnetic filter.

24) A system relating to biological sterilization of at least one medical item comprising:
a) plasma generator means for generating sterilizing plasma comprising sterilization constituents capable of such biological sterilization and surface-harming constituents capable of harming surfaces of the at least one medical item to be sterilized;
b) interactor means for promoting interaction between such sterilizing plasma and the at least one medical item to be sterilized; and
c) electromagnetic filter means for electromagnetically filtering such surface-harming constituents from such sterilization constituents;
d) wherein said electromagnetic filter means selectively passes, to surfaces of the at least one medical item, a portion of such sterilization constituents while suppressing passage, to the surfaces of the at least one item, a portion of such surface-harming constituents;
e) wherein the at least one medical item is sterilized by exposure to such sterilization constituents capable of biological-sterilization; and
f) wherein surface harm to the at least one medical item is reduced by suppressing passage of such surface-harming constituents to the surfaces of the at least one medical item.
